(19) United States
(12) Patent Application Publication
(10) Pub. No.: US 2009/0318982 A1
(43) Pub. Date: Dec. 24, 2009

(54) METHODS OF INJECTING CALCIUM BASED NEUTRAL AND BIORESORBABLE BONE GRAFTS

(75) Inventors: Francois GENIN, Berkeley, CA (US); Ping LUO, Berkeley, CA (US)

Correspondence Address: MORRISON & FOERSTER LLP, 425 MARKET STREET, SAN FRANCISCO, CA, 94105-2482, US

(73) Assignee: Berkeley Advanced Biomaterials, Inc., Berkeley, CA (US)

(21) Appl. No.: 12/552,236

(22) Filed: Sep. 1, 2009

Related U.S. Application Data
(62) Division of application No. 09/862,206, filed on May 21, 2001.

(51) Int. Cl. A61B 17/58

(52) U.S. Cl. 606/86 R

(57) ABSTRACT

An injectable and moldable putty comprising biodegradable calcium-based compounds including calcium sulfate, hydroxyapatite, and tricalcium phosphate is invented. The putty hardens into a solid body when mixed with water, saline, serum, or other neutral aqueous solutions. The hardening time of the putty can be tailored in order to meet the specific requirements of various dental or orthopedic applications. The pH of the putty is neutral during and after mixing. The invented putty may be used as bone graft, bone implant, or implantable drug delivery device.

### CROSS-REFERENCE TO RELATED APPLICATION

This application is a Divisional application of U.S. patent application Ser. No. 09/862,206, entitled CALCIUM BASED NEUTRAL AND BIORESORBABLE BONE GRAFT, filed on May 21, 2001, which is incorporated herein by reference in its entirety for all purposes.

### TECHNICAL FIELD

This invention relates to injectable, moldable, and bioresorbable bone grafts containing calcium sulfate and calcium phosphate cementing powder and cementing reagents including neutral aqueous solutions and buffers, useful in dental and bone cements, bone graft materials, bone tissue substitutes, bone void fillers, and drug release carriers.

### BACKGROUND OF THE INVENTION AND DESCRIPTION OF RELATED ART

The use of synthetic biocompatible, bioresorbable, injectable or moldable putty or cement implant materials is increasing in orthopedic, plastic and dental surgery applications. Such materials are typically needed to add bone mass or replace damaged bone tissue at the surgical site (e.g. bone loss caused by periodontal disease, ridge augmentation, bone defect or cavity due to trauma, cancer/disease, or surgery and spinal fusion). After being implanted, the bone substitute begins to resorb and is replaced by new bone as a result of the action of bone cells. In orthopedic surgery, autografts are commonly used for bone repair. Unfortunately, such procedure requires second site surgery which increases the burden on the patient and can delay his/her recovery.

When properly processed and implanted, autografts, allografts, xenografts, and demineralized bone grafts typically show excellent biocompatibility and can sometimes induce bone growth. Their main disadvantage is related to the potential of transmitting diseases such HIV, hepatitis and recently discovered ailments caused by proteins (e.g. mad cow). Proteins or other organic substances may not always be removed by chemical processes or sterilization as a result of internal porosity. This has the potential for causing adverse immunological reactions resulting in inflammation or rejection after implantation and for spreading genetic defects in the future.

Calcium sulfate hemihydrate also commonly referred as to Plaster of Paris is bioresorbable. In 1892, Dreesmann used it for the first time as a filler material in osseous defects. It has since been used in medicine for many orthopedic, plastic surgery and dental applications. This material is moldable and can harden in less than 20 minutes making it easy and convenient to use. However, calcium sulfate in its pure form presents a disadvantage for bone substitute applications. Researchers have determined that calcium sulfate takes 3 to 6 weeks to resorb. Such a high rate of resorbtion in the body does not match the natural rate of growth of new bone that in turn can leave a void at the implanted site a few month after the surgery. In addition, pure calcium sulfate is acidic and may cause soft tissue damage or irritation that lead to inflammatory reactions after implantation and during resorbtion.

Other materials for repair of bone defects such as metal, non-resorbable ceramic, and polymers, for example, silicone, Proplast, or methylmethacrylate are often encapsulated by scar tissue. This leads to a significant probability of implant infection or rejection.

The human body is composed of 65% to 70% calcium phosphate minerals including tri-calcium phosphate and hydroxyapatite. Hydroxyapatite is more stable than other calcium phosphates. Hydroxyapatite and tricalcium phosphates are ideal candidates for human bone hard tissue replacements. Many inventions have focused on the preparation and application of hydroxyapatite and tricalcium phosphate bone cements. These inventions and other clinical studies have proven that hydroxyapatite, tricalcium phosphates and calcium sulfate materials have excellent biocompatibility properties and are safe for human implant applications.

A number of biocompatible and bioresorbable bone graft substitutes, bone cements, and putties have been reported in the literature. Inventions on this topic typically describe compositions that lead to a paste that can harden after mixing a solid (generally in the form of powder) with a liquid. The powder is a calcium-based material and the liquid is often a polymer or sometimes an aqueous solution.

Calcium phosphate self-setting bone cements for bone-repair have been described in U.S. Pat. Nos. 5,997,624, 5,976,234, 5,954,867, and 5,525,148. In these patents, the cement comprises tetracalcium phosphate, which converts to hydroxyapatite after setting. Anhydrous dicalcium phosphate and dicalcium phosphate dihydrate may be combined. The tetracalcium phosphate is prepared and maintained under substantially anhydrous conditions prior to its contact with the aqueous medium. The cement sets within 15 to 30 minutes and fully converts to a solid mass of hydroxyapatite in vivo within 4 to 6 hours. However, several disadvantages and risks are associated with this type of cement. First, the setting time is long which makes inconvenient for use in the surgical room. Second, incomplete in vivo conversion into hydroxyapatite may occur, resulting in inconsistent post-operation implant chemical compositions. The resorbtion rate is therefore unpredictable. Finally, a pH above 12.5 makes this cement potentially harmful to surrounding soft tissues.

Other cements such as calcium sulfate with or without fillers of calcium (sodium or potassium) phosphate ceramics have been developed. In U.S. Pat. No. 5,281,265, compositions of resorbable cements are described. The cementing components selected from the group consisting of calcium sulfate-containing components, calcium succinate, calcium malate, calcium malonate, calcium maleate, hydrates thereof and mixtures thereof. The other setting components are polyfunctional carboxylic acids and water-soluble dibasic phosphate salts. When calcium sulfate powder is mixed with citrate in water, the calcium sulfate salt dissolves to provide calcium ion to form a less soluble calcium citrate salt for cement formation. After hardening, it is used as bone graft for implantation. The hardened cement has a surface, which is substantially neutral or alkaline in character. In U.S. Pat. No. 5,149,368, a powder mixture of calcium phosphates or tetracalcium phosphate alone will harden when mixed with cementing setting reagents. The cement is slightly acidic at the beginning of the setting. After setting, the pH raises up to 7. These types of cements are useful when placed at the surgical site after complete hardening. However, their characteristic limits their use when direct injection of the paste or putty into the surgical site is required before hardening takes place.

In U.S. Pat. No. 5,679,723, absorbable or resorbable mixtures of aliphatic polyesters and calcium containing bone regenerating compounds such as powdered, non-fibrous calcium phosphates are described. This invention focused on the description of a liquid, low melt, injectable biocompatible composite comprised of a polymer and a calcium-based material which exhibit improved absorption characteristics.

In U.S. Pat. No. 6,005,162, the invention relates to the preparation of calcium phosphate minerals for physiological applications in which phosphoric acid substantially free of uncombined water is combined with a calcium source and neutralizing anions. The anions include at least one of carbonate, phosphate and hydroxide in an amount sufficient to substantially neutralize said phosphoric acid with water such that a flowable composition capable of setting into a calcium phosphate mineral is produced.

### OBJECTIVE AND DISCLOSURE OF THE INVENTION

The first objective of the present invention is to design the composition of an injectable, moldable, biocompatible and bioresorbable bone graft in the form of a putty or cement that is neutral during and after mixing and setting. The second objective is to design a putty or cement that can harden in both dry and wet environment at the implant site. The third objective of the invention is to design the composition of the bone graft that remains neutral during resorbtion. The fourth objective is to design the composition of the putty or cement with desired setting times.

As will become apparent, preferred features and characteristics of one aspect of the invention are applicable to any other aspects of the invention.

In one aspect, the invention provides a method to form or to inject a bone graft at the surgical site.

In a preferred embodiment, the implant is formed by two groups of cementing components. One group is the cementing powder and the other group is the cementing reagent. The cementing powder is a mixture of calcium sulfate, hydroxyapatite, and tricalcium phosphates. In another preferred embodiment, the calcium sulfate, hydroxyapatite, and tricalcium phosphates are either amorphous or crystalline. The particle size of individual phases is not limited in this invention. In another preferred embodiment, the hydroxyapatite and calcium phosphate are dense or porous granules. In another preferred embodiment, the calcium sulfate is anhydrous and has not been heat-treated or thermally annealed above 700 degrees C. In another preferred embodiment, the cementing reagent is neutral with a pH value ranging from 6.5 to 7.5. The cementing reagent can be a single or a mixture of more than one of the following neutral reagents such as distilled water, saline solutions, serum solutions, sodium chloride solutions, blood, and a mixture thereof depending on the desired setting time for the particular surgical needs. In another preferred embodiment, the cementing reagent is a buffer solution. In other preferred embodiments, the buffers are PBS (pH=7.2 or 7.4), Phosphate Buffer (pH=6.8 or 7.2), SSC (pH=7.0), and SSPE (pH=7.2).

In another preferred embodiment, the cementing powder contains at least 30% of calcium sulfate. In another preferred embodiment, when the cementing powder is mixed with water to form a putty and then to contact with blood before hardening, the pH of the putty remains neutral. In another preferred embodiment, the setting time can be tailored by changing the ratio of calcium sulfate to calcium phosphates in the cementing powder.

In another preferred embodiment, neutral, weak acidic, and/or weak basic salts can be added to the cementing dry powder to modify the setting time. Buffers may added as needed to keep the paste or putty neutral during mixing and setting.

In another preferred embodiment, the invention includes mixing dry powder with neutral water or buffers to form a paste or putty that can be worked to form an desired object or injected directly into the surgical site. In another preferred embodiment, the implant site can be dry or wet.

In another preferred embodiment, the paste or putty is shaped and/or molded into an object before it hardens with a mold, a punch tool, or a stick in order to produce pores or holes or in order to form desired shapes before implantation.

In other preferred embodiment, other bioresorbable compounds, non-resorbable compounds, and biomolecules can be incorporated into the cementing powder to treat patients of various ages.

### SUMMARY OF THE INVENTION

This invention pertains to a calcium sulfate cement or putty containing hydroxyapatite and/or calcium phosphates. The cement is neutral before, during, and after setting. This provides excellent biocompatibility with human tissue. Changing the ratio of calcium sulfate, hydroxyapatite, and calcium phosphates in the mixture allows to change the resorbtion rate of the implant. Once the cementing powder is mixed with cementing reagents, it becomes a paste or a putty. The pH of the paste or the putty remains neutral before and during setting. Neutral and/or inorganic salts can be added into the cementing powder to tailor the setting time. Neutral buffers from pH 6.5 to pH 7.4 can be also used as cementing or setting reagents to adjust the pH value of the paste or the putty to neutral. The paste or the putty will harden between 2 to 30 minutes at temperatures between 10 and 40 degrees C. The hardening time is a function of the composition of the cementing powder and the chemistry of the setting reagents.

The resorbtion rate of calcium-based implants in the human body is known to vary upon phase and composition. It can also change from patient to patient. If implanted under the same surgical conditions and in the same patient, hydroxyapatite resorbs slower than tricalcium phosphate and tricalcium phosphate resorbs slower than calcium sulfate. By tailoring the ratio of the mixture of these three solids, the resorbtion rate and resorbtion profile can be tailored. For example, the addition of hydroxyapatite, and beta-tricalcium phosphate to calcium sulfate will slow down the implant resorption process and will help support bone regeneration at the site for a longer period of time.

The morphology of the calcium sulfate, hydroxyapatite, calcium phosphates, or other calcium-based materials (such as calcium carbonate, calcium citrate, and calcium acetate) in this invention can be varied depending on the required resorbtion rate. In general, the larger the particles, the slower the resorbtion rate. Sintered granules have slower resorbtion rate than non-sintered granules or amorphous granules. Porous granules will resorb faster than dense ones.

Sometimes, the surgical site has a complex geometry. It can be located behind other organs. To minimize collateral damage, it is often preferable to inject, fill, or patch the putty directly into the void before hardening occurs. The putty then hardens in vivo after injection. In order to mitigate potential irritation or inflammatory reactions or minimize harm to the tissue, it is preferable that the paste or putty be neutral. In this invention, the cementing powder mixture contains at least 30 wt % calcium sulfate and at least 20 wt % calcium phosphates. The cementing powder is not pure calcium sulfate. When mixed with distilled water, the pH is neutral. When mixed with saline solution or water (pH=6.5 to 7.5) or sodium chloride solution, or blood, the pH also remains neutral. Neutral buffers can also be used as cementing reagents. For example, PBS buffer (pH=7.2 or 7.4), Phosphate Buffer (pH=6.8 or 7.2), SSC (pH=7.0), and SSPE (pH=7.2). The neutral buffers used as liquid cementing reagents are selected so that the ingredients are biocompatible and bioresorbable. Since the setting time varies with the composition of the cementing powder and the type of the cementing agent, a combination of the above cementing agents can be used to achieve the desired setting time for the surgical needs.

In order to treat patients with different ages, various resorbtion rates and various physical properties are needed for the bone grafts. The cementing powder in this invention can be mixed with other biocompatible (bioresorbable and non-resorbable) materials to form a composite to enhance physical, chemical, and mechanical properties, osteoinductive properties, and other physical and biochemical properties. These include collagen, demineralized bone matrix, hyaluronic acid and derivatives thereof, polyanhydrides, polyorthoesters, polyglycolic acid, polylactic acid, and copolymers thereof, polyesters of alpha-hydroxycarboxylic acids, poly(L-lactide) (PLLA), poly(D,L-lactide) (PDLLA), polyglycolide (PGA), poly(lactide-co-glycolide (PLGA), poly(D,L-lactide-co-trimethylene carbonate), and polyhydroxybutyrate (PHB), polyanhydrides, poly(anhydride-co-imide) and co-polymers, bioactive glass compositions, dextrans, polyethylene, polymethylmethacrylate (PMMA), carbon fibers, polyvinyl alcohol (PVA), poly(ethylene terephthalate) polyamide, titania, ziconia, alumina, yttria, silica, and mixtures thereof.

Basic and acidic proteins, peptides, DNAs, RNAs, plasmids, antibiotics such as gentamycin, trobamycin and ciprofloxacin, anti-cancer agents and chemicals such as doxorubicin can be incorporated into the cementing powder or the cementing liquid reagents in this invention to form delivery devices for gene-therapy and chemotherapy applications. The above biomolecules can be incorporated directly into the cementing powder or cementing liquid during manufacture. They can be also packed individually and included separately. The additives can be directly mixed into the cementing powder or cementing reagent during surgery before hardening.

The term “cementing powder” refers to biodegradable powder mixture that play a role when mixed with a liquid to form a putty or cement.

The term “cementing reagents” refers to biodegradable liquid reagents that play a role when mixed with a powder to form a putty or cement.

The term “putty” refers to an injectable, moldable, and workable paste containing cementing powder and cementing reagents before hardening into a cement.

The term “bone graft” refers to a hardened putty worked into an implant.

The term “neutral reagents” refer to a pH value of the reagents between 6.5 and 7.5.

The term “neutral putty” refers to the pH value of the putty between 6.5 and 7.5.

The term “neutral buffers” include all liquid containing biocompatible and biodegradable ingredients that are neutral between 6.5 and 7.5. The buffers balances the pH to neutral when mixing with cementing powder or other cementing reagents that is slightly acidic or basic. The neutral buffers can be used alone as cementing reagents.

### EXAMPLE OF THE INVENTION

### Example 1

### Fabrication of Cementing Powder with Desired Setting Times

Calcium sulfate anhydrous (CaSO₄) and calcium phosphates including hydroxyapatite and tricalcium phosphate are mixed into ratios of 0.44, 0.61 and 1.2 by weight. The ratio of hydroxyapatite and tricalcium phosphate in this study is 2.33. The workable time and setting time are described in the following table. The higher the calcium sulfate anhydrous (CaSO₄) to calcium phosphates ratio, the shorter the workable time, the longer the setting time, and the shorter the time required for complete hardening.

TABLE 1
Comparison of calcium sulfate anhydrous (CaSO₄) to calcium
phosphates ratios to the required the hardening time. The setting
agent is distilled water (pH = 7).

| Cementing Powder | | | Total Time |
|---|---|---|---|
| Concentrations CaSO₄ to | | | Required for |
| Calcium Phosphates | Workable Time | Setting Time | Hardening |
| (ratio) | (minutes) | (minutes) | (minutes) |
| 0.44 | 15 | 15 | 30 |
| 0.61 | 5 | 15 | 20 |
| 1.2 | 2 | 2 | 4 |

### Example 2

### Fabrication of Cementing Reagents To Control Setting Times

Calcium sulfate anhydrous (CaSO₄) and calcium phosphates including hydroxyapatite and tricalcium phosphate are mixed into ratios of 0.44 and 1.2 by weight. The cementing liquid reagents are distilled water (pH=6.5 or 7), saline water (pH=6.5), PBS buffer (pH=7.2), saturated NaCl solution (pH=7), and blood (pH=7), respectively. As seen in Tables 2 and 3, the workable time and required setting time are shortened when the PBS solution and the saturated sodium chloride are used as cementing reagents. On the other hand, when the calcium sulfate anhydrous (CaSO₄) to calcium phosphates ratio is 1.2, the setting time is longer when the PBS buffer is used than when distilled water or saturated sodium chloride solution are used.

TABLE 2
Hardening time for various cementing reagents, when the calcium
sulfate anhydrous (CaSO₄) to calcium phosphates ratio is 0.44.

| | | | Total Time |
|---|---|---|---|
| Cementing | Workable Time | Setting Time | Required |
| Reagents | (minutes) | (minutes) | (minutes) |
| Water | 15 | 15 | 30 |
| PBS Buffer | 5 | 5 | 10 |

TABLE 3
Hardening time for various cementing reagents, when the
calcium sulfate anhydrous (CaSO₄) to calcium phosphates
ratio equals 1.2.

| | | | Total Time |
|---|---|---|---|
| Cementing | Workable Time | Setting Time | Required |
| Reagents | (minutes) | (minutes) | (minutes) |
| Water | 2 | 2 | 4 |
| Saline Water | 3 | 7-10 | 10-13 |
| PBS Buffer | 1 | 4 | 5 |
| Saturated | 1 | 1 | 2 |
| NaCl | | | |
| Solution | | | |
| Blood | 60 | >90 | >150 |

### Example 3

### Repeatability of the Setting Time and the Methods for Implantation

The hardening time is consistent and repeatable when PBS buffer is used. When the PBS buffer is used as cementing reagent, the paste or the putty can harden when directly injected into water or blood. The paste or the putty will harden in both wet and dry environments. The total hardening time remains the same (i.e. 5 minutes). This characteristic ensures that such bone graft can be injected directly into a cavity or a wound when blood is present.

TABLE 4
Repeatability of hardening time for various implantation methods and media
and for a calcium sulfate anhydrous (CaSO₄) to calcium phosphates
ratio equal to 1.2.

| | | | | Total Time | | Observations |
|---|---|---|---|---|---|---|
| | PBS | | Setting | required for | | after 30 |
| Cementing | Buffer | Working | Time | implantation | | minutes and |
| Powder | (pH = 7.2) | Conditions | (minutes) | (minutes) | Methods | 1 hour |
| 5 cc (2.6 g) | 2 cc | 1 minute | 4 | 5 | Place in | hard in |
| | | to make a | | | water after | water |
| | | sphere | | | hardening | |
| 5 cc (2.6 g) | 2 cc | 1 minute | 4 | 5 | Place in | hard in |
| | | to make a | | | water after | water |
| | | sphere | | | hardening | |
| 5 cc (2.6 g) | 2 cc | 1 minute | 4 | 5 | Place in | hard in |
| | | to make a | | | water after | water |
| | | sphere | | | hardening | |
| 5 cc (2.6 g) | 2 cc | 1-2 | 0 | 1 | Inject into | hard in |
| | | minute to | | | water | water after |
| | | prepare a | | | directly | 5 minutes |
| | | paste | | | | |
| 5 cc (2.6 g) | 2 cc | 1-2 | 0 | 1 | Inject into | hard in |
| | | minute to | | | blood | water after |
| | | prepare a | | | directly | 5 minutes |
| | | paste | | | | |
| 5 cc (2.6 g) | 2 cc | 1 minute | 0 | 1 | Place | hard in |
| | | to prepare | | | directly in | water after |
| | | a ball | | | water | 5 minutes |
| | | | | | when soft | |
| 5 cc (2.6 g) | 2 cc | 1 minute | 0 | 1 | Place | hard in |
| | | to make a | | | directly in | water after |
| | | sphere | | | water | 5 minutes |
| | | | | | when soft | |

### Example 4

### Compositions of Several Neutral Cementing Reagents

1) Distilled Water: 6.5≦pH≦7.5

2) PBS Buffer: 0.017M KH₂PO₄, 0.05M Na₂HPO₄, 1.5M NaCl, pH=7.4.

Or 0.144 g/l KH₂PO₄, 9 g/l NaCl, 0.795 g/l Na₂HPO₄.7H₂O, pH=7.2

3) Phosphate Buffer: 70 g/l calcium phosphate monobasic (Ca(H₂PO₄)₂.H₂O, 131.3 g/l calcium phosphate dibasic (CaHPO₄ or CaHPO₄.2H₂O), pH=6.8

### REFERENCES CITED

### Referenced by

### U.S. Patent Documents

### 5,824,087 Oct. 20, 1998 Aspden

### 5,522,893 Jun. 4, 1996 Chow

### 5,997,624 Dec. 7, 1999 Chow

### 6,005,162 Dec. 21, 1999 Constantz

### 5,514,137 May 7, 1996 Coutts

### 6,206,957 Mar. 27, 2001 Driessens

### 4,843,112 Jun. 27, 1989 Gerhart

### 5,085,861 Feb. 4, 1992 Gerhart

### 6,027,742 Feb. 22, 2000 Lee

### 5,149,368 Sep. 22, 1992 Liu

### 5,218,035 Jun. 8, 1993 Liu

### 5,262,166 Nov. 16, 1993 Liu

### 5,281,265 Jan. 25, 1994 Liu

### 5,462,722 Oct. 31, 1995 Liu

### 5,858,318 Jan. 12, 1999 Luo

### 5,296,026 Mar. 22, 1994 Monroe

### 5,356,629 Oct. 18, 1994 Sander

### 5,366,507 Nov. 22, 1994 Sottosanti

### OTHER REFERENCES

Anson, Compend. Contin. Educ. Dent. 21(5), p. 365-70, p. 372-3, 376 (2000).
Baker, An In Vivo Evaluation of Artificial Bone Constructs, Washington D.C., 1992 (Orthopedic Research Society).
Block, J. Oral Maxillofacial Surgery 46, p. 420-425 (1988).
Brown, A New Calcium Phosphate, Water Setting Cement in Cements Research Progress 1986, edited by P. W. Brown (American Ceramic Society, Westerville, Ohio, 1988), p. 352-379.
Chow, Self-Setting Calcium Phosphate Cements, Mat. Res. Soc. Symp. Proc. 179 (1991).
Chow, X-ray Diffraction and Electron Microscopic Characterization of Calcium Phosphate Cement Setting Reactions, IADR, 1987.
Chow, Adv. Dent. Res. 2(1), p. 191-184 (1988).
Chow, Journal of The Ceramic Society of Japan 99, p. 954-964 (1991).
Constantino, Arch. of Otolaryngology—Head & Neck Surgery 117, 379-394 (1991).
Constantino, Plastic and Reconstructive Surgery 90, p. 174-185 (1992).
Constantz, Pilot Investigations of a Calcium Phosphate Cement in a Rabbit Femoral Canal Model and a Canine Humeral Plug Model, 1991.
Constantz, Science 267 (Mar. 24), p. 1796-1799 (1995).
Costantino, Evaluation of a New Hydroxyapatite Cement: Cranioplasty in a Cat Model, Toronto, Canada, 1989.
Costantino, Evaluation of a New Hydroxyapatite Cement: Basic Chemistry and Histology, Toronto, Canada, 1989.
Damien, J. Applied Biomaterials 2, p. 187-208 (1991).
Daniels, J. Applied Biomaterials 1, p. 57-78 (1990).
DeRijk, Clinical Evaluation of a Hydroxyapatite Precipitate for the Treatment of Dentinal Hypersensitivity, Biomedical Engineering v. Recent Developments, Subrata
Saha, (Pergamon Press: New York), p. 336-339 (1986).
Driskell, J. Biomed. Mat. Research 6, p. 345-361 (1972).
Friedman, Evaluation of a New Hydroxyapatite Cement: Obliteration and Reconstruction of the Cat Frontal Sinus, Toronto, Canada, 1989.
Friedman, Arch. of Otolaryngology—Head & Neck Surgery 117, p. 385-389 (1991).
Fujikawa, J. Dent. Materials 10, p. 45-57 (1995).
Gao, J. Bio. Mat. Research 32, p. 505-512 (1996).
Grote, Arch Otolaryngology 110, p. 197-199 (1984).
Grunninger, J. Dent. Res. 63 (Special Issue) (1984).
Hadjipavlou, Spine 25, p. 10-5 (2000).
Hanker, J. Dent. Research 66, 1144 (1987).
Hiatt, J. Periodontal 43, p. 373-380 (1972).
Horioglu, Transactions of the 21st Annual Meeting for the Society for Biomaterials, San Francisco, Calif., Mar. 18-22, p. 198 (1995).
Ioku, Biomed. Mater. Eng. 3, p. 137-45 (1993) Johnson, J. Orthop. Review 14, p. 351-369 (1996).
Kelly, Clin Orthop. 382, p. 42-50 (2001).
Kenney, The Use of a Porous Hydroxyapatite Implant in Periodontal Defects, J. Periodontal 59, p. 67-72 (1988).
Kent, J. Oral Maxillofacial Surgery 41, p. 629-642 (1983).
Kim, Oral Surg. Oral Med. Oral Pathol. Oral Radiol. Endoc. 88 (1), p. 22-25 (1999).
Le Maitre, Silicate Industries 9-10, p. 141 (1987).
LeGeros, Apatitic Calcium Phosphates: Possible Dental Restorative Materials, 1482 (1982).
Link, Composite of Calcium Phosphate Cement and Genetically Engineered Protein Bioadhesive, 1991.
Lu, J. Dent. Research 67, p. 352 (1988).
Luo, Materials Science & Engineering C₃, p. 75-78 (1995)
Luo, Biomaterials 17, p. 1959-64 (1996).
Luo et al., Hydroxyapatite drug delivery implant for treating bone infections, Kamuela, Hi., 2000.
Luo et al., Synthetic Calcium Phosphate-based Skeletons, Toronto, p. 627 (1996).
Luo et al., Protein binding potential of biocompatible ceramic hydroxyapatite microspheres, San Diego, 1998.
Matsuya, Effects of pH on the Reactions of Tetracalcium Phosphate and Dicalcium Phosphate, IADR 1991.
Mirtchi, Biomaterials 10, p. 475-480 (1989).
Misch, Int. J. Oral Implant 4, 49-58 (1987).
Miyazaki, Polymeric Calcium Phosphate Cements, IADR 1990.
Miyazaki, Chemical Change of Hardened PCA/CPC Cements in Various Storing Solutions, J. of the Jap. Soc. for Dent. Mat. and Devices 11, 2, (1992).
Muller-Mai, Journal of Bio. Mat. Research 29, p. 9-18 (1995).
Peltier, The Use of Plaster of Paris to Fill Large Defects in Bone: a preliminary report 1959, Clinical Orthop. 382, p. 3-5 (2001)
Pham et al., Preparation and Characterization of Novel Drug Delivery Implants for the Treatment of Bone Infections, New Orleans, 1999.
Piecuch, Dental Clinics of North America 30, p. 291-305 (1986).
Salyer, Plas and Recon Surgery 84, p. 236-244 (1989).
Sanin, Particle Size Effects on pH and Strength of Calcium Phosphate Cement, 1991.
Schreiber, J. Dent. Research 67, p. 959-63 (1988).
Setya, Periodontal Clin. Investigation 21, p. 5-14 (1999).
Shindo, Arch. of Otolarynology—Head and Neck Surgery, 199, p. 185-190 (1993).
Sugawara, J. of Jap. Soc. for Dent. Mat. and Devices 8, 2 (1989).
Sugawara, J. Dent. Research 66, p. 296 (1987).
Sugawara, Histopathological Reactions of a Calcium Phosphate Cement Root Canal Filler, 1991.
Sugawara, J. Hard Tissue Biology 4, p. 1-7. (1995).
Tay, Orthop. Clin. North America 30, p. 615-23 (1999).
TenHuisen, Journal of Bio. Mat. Res., 29, p. 803-810 (1995).
Tian, A Novel Chondrocyte-Seeded Hydroxyapatite-Collagen Scaffold for Cartilage Repair, Fifth World Biomaterials Congress, 1996.
Verheyen, J. Bio. Mat. Research 27, p. 433-444 (1993).
Waite, J. Oral Maxillofacial Surgery 44, p. 349-352 (1986).
Zide, J. Oral Maxillofacial Surgery 45, p. 481-486 (1987).

What we claim is:

1: A method for implanting a biocompatible and injectable bone graft comprising:
mixing a cementing powder and a cementing reagent to form a putty with a neutral pH, wherein said cementing powder comprises one or more calcium phosphates in a form that is insoluble and at least 30% by weight calcium sulfate and said cementing reagent comprises a neutral buffer;
injecting the putty into a bone void; and
allowing the putty to set inside the bone void, wherein the neutral buffer the putty at a neutral pH during mixing, injecting into a bone void, and setting inside of a bone void.

2: The method of claim 1, wherein said putty at an ambient temperature range is injectable and workable for a period of time from about 1 minute to about 15 minutes in a dry or a wet environment, and hardens from between about 3 minutes to about 30 minutes, wherein the ambient temperature range of said injectable and workable period and said hardening period is between 10° C. and 40° C. Minutes.

3: The method of claim 1, wherein the one or more calcium phosphates are selected from the group consisting of hydroxyapatite, alphatricalcium phosphate, beta-tricalcium phosphate, tetra-calcium phosphate, octacalcium phosphate, di-calcium phosphate, calcium hydro-phosphate, brushite and monetite.

4: The method of claim 3 wherein the one or more calcium phosphates comprise hydroxyapatite and beta-tricalcium phosphate.

5: The method of claim 1 wherein the cementing powder further comprises a calcium-based compound-selected from the group consisting of calcium carbonate, calcium citrate, calcium acetate, calcium oxide, calcium hydroxide and apatites.

6: The method of claim 5 wherein the apatites are selected from the group consisting of fluorapatite and carbonate apatite.

7: The method of claim 1 wherein the neutral buffer comprises a phosphate buffer.

8: The method of claim 7, wherein the phosphate buffer neutralizes the bone graft while providing the optimum resorption rate of the bone graft.

9: The method of claim 1 wherein the calcium sulfate is in the anhydrous, hemihydrates, or dihydrates in alpha, beta, or gamma phases.

10: The method of claim 1 wherein the cementing reagent has a pH value from about 6.5 to about 7.5 and is selected from the group consisting of distilled water, saline water, serum water, sodium chloride solution, sodium phosphate solution, blood, and buffer solutions.

11: The method of claim 1 wherein the bone graft has a resorption rate that is tailored by changing the proportion of the calcium sulfate to the one or more calcium phosphates.

12: The method of claim 1 wherein the bone graft has a resorption rate that is tailored by varying the crystallinity of the calcium sulfate and the one or more calcium phosphates.

13: The method of claim 1 further comprising a biocompatible material that enhances a physical, chemical, or mechanical property of the bone graft.

14: The method of claim 13 wherein the biocompatible material is bioresorbable.

15: The method of claim 14 wherein the biocompatible materials are selected from the group consisting of collagen, fibrin, demineralized bone matrix, hyaluronic acid and derivatives thereof, polyanhydrides, polyorthoesters, polyglycolic acid, polylactic acid and copolymers thereof, polyesters of alpha-hydroxycarboxylic acids, polyglycolide (PGA), poly(L-lactide) (PLLA), poly(D,L-lactide) (PDLLA), poly(lactide co-glycolide (PLGA), poly(D,L-lactide-co-trimethylene carbonate), polyhydroxybutyrate (PHB), polyanhydrides, poly(anhydride-co-imide) and co-polymers thereof, bioactive glass compositions and combinations thereof.

16: The method of claim 13 wherein the biocompatible material is non-resorbable.

17: The method of claim 16 wherein the biocompatible materials are selected from the group consisting of dextrans, polyethylene, polymethylmethacrylate (PMMA), carbon fibers, polyvinyl alcohol (PVA), polyethylene terephthalate) polyamide, titania, ziconia, alumina, yttria, and silica.

18: The method of claim 1 further comprising a biomolecule.

19: The method of claim 18 wherein the biomolecule is selected from the group consisting of acidic or basic proteins, bone morphogenetic proteins, peptides, DNAs, RNAs, antibiotics, anti-cancer agents; and chemicals for gene therapy or chemotherapy.

20: The method of claim 1 wherein the one or more insoluble calcium phosphates comprise at least 20% by weight of the bone graft.

21: The method of claim 1 wherein the one or more insoluble calcium phosphates are amorphous or crystalline.

22: The method of claim 1 wherein the putty further comprises a magnesium phosphate in an apatite form.
